(19) United States
(12) Patent Application Publication
(10) Pub. No.: US 2009/0226495 A1
(43) Pub. Date: Sep. 10, 2009

(54) NANODIAMOND ENHANCED EFFICACY

(76) Inventors: Salvatore Charles PICARDI, Souderton, PA (US); Ali Razavi, Dallas, PA (US)

Correspondence Address: ZALE Patent Law, 121 Old Orchard Road, Clarks Summit, PA, 18503-2053, US

(21) Appl. No.: 12/399,844

(22) Filed: Mar. 6, 2009

Related U.S. Application Data
(63) Continuation of application No. PCT/US2007/016194, filed on Jul. 17, 2007.
(60) Provisional application No. 61/034,173, filed on Mar. 6, 2008.
(60) Provisional application No. 61/118,281, filed on Nov. 26, 2008.

(51) Int. Cl. A61K 9/00; A61K 9/14; A61K 9/02; A61K 33/40

(52) U.S. Cl. 424/404; 424/489; 424/436; 424/402; 424/613; 977/906

(57) ABSTRACT

The present invention is directed to attaching drugs and other functional groups to surfaces of nano-sized diamonds (NDs) to enhance the efficacy of drugs and other substances. The method involved enhancing the efficacy of a drug having an active site by acquiring a plurality of nanodiamond (ND) particles having a plurality of carbon chain surface molecules on its surface. Intermediate amine entities are covalently attached to the surface molecules of the ND particles. These are then replaces with said drug molecules such that the active sites of said drug molecules point away from the ND particle exposing them for enhanced activity and enhanced drug efficacy. The efficacy of antimicrobial drugs are enhanced, however, this may be used with many different types of drugs.

### CROSS REFERENCE TO RELATED APPLICATIONS

This patent application is A Continuation-In-Part of earlier-filed provisional patent application Ser. No. 61/034,173 filed Mar. 6, 2008, by S. Charles PICARDI and Ali RAZAVI. This application also claims priority from Ser. No. 61/118,281 entitled “Nanodiamond Enhanced Drugs” by Salvatore Charles PICARDI and Ali RAZAVI filed Nov. 26, 2008. This patent application also incorporates by reference Ser. No. 60/831,438 entitled “Biofunctional Articles For Personal Care Applications and Method of Making the Same” by Ali RAZAVI filed Jul. 18, 2006, and PCT patent application PCT/US2007/016,194 “Multifunctional Articles And Method For Making The Same” by Ali RAZAVI filed Jul. 17, 2007 as if both were set forth in their entirety herein.

### BACKGROUND OF THE INVENTION

1. Field of the Invention

The present invention relates to a substance and method of enhancing the efficacy of drugs and more specifically a substance and method of increasing the efficacy of antimicrobials (AMs).

2. Discussion of Related Art

There has always been a need to increase the efficacy of various drugs and preparations. One such class of drugs to be used as an example in this application are the antimicrobials (AMs). AMs are typically used to limit or stop the spread of unwanted microbes. AMs are effective when used in the proper level but lose their ability as the concentration drops below a critical concentration. This may be due to the fact that enough active sites on antimicrobial molecules must make contact with active sites on critical molecules inside of a microbe to cause the microbe to have an effect.

AMs are typically used in an aqueous solution inside of a person or animal. Molecules flowing in a solution are randomly dispersed and oriented. Also, since AMs flow in solution to attach to active sites on the microbe, it is important to have a large amount of the AMs in solution, increasing the local concentration and the potential of attaching to an active site on a microbe molecule.

Currently, there is a need for AMs that are more soluble in a fluid, and attach more readily to active sites on molecules of microbes to inactivate the microbes.

### SUMMARY OF THE INVENTION

One embodiment of the present invention is a method of enhancing efficacy of a drug having an active site, comprising the steps of:
a) acquiring a plurality of nanodiamond (ND) particles having a plurality of carbon chain surface molecules on its surface, the ND particles having a diameter of less than 10 nanometers;
b) covalently attaching a plurality of intermediate amine entities to the surface molecules of the ND particles, and
c) replacing at least a portion of the attached intermediate amine entities attached to the surface molecules of the ND particles with said drug molecules such that the active sites of said drug molecules point away from the ND particle exposing them for enhanced activity and enhanced drug efficacy.
   This method is very effective in enhancing the efficacy of antimicrobial agents; however other drugs may be employed.

### OBJECTS OF THE INVENTION

It is an object of the present invention to provide an antimicrobial which is more potent than previous antimicrobials.

It is another object of the present invention to provide an antimicrobial which is more soluble than prior art antimicrobials.

It is another object of the present invention to provide a method of amplifying the effect of an antimicrobial in-situ.

It is another object of the present invention to provide a method of holding antimicrobial molecules in an orientation to maximize interaction with microbes.

It is another object of the present invention to provide a method for locally increasing the effective concentration of an antimicrobial while keeping the overall concentration constant.

### BRIEF DESCRIPTION OF THE DRAWINGS

The advantages of the instant disclosure will become more apparent when read with the specification and the drawings, wherein:

FIG. 1 is a schematic illustration of how molecules react under normal prior art conditions.

FIG. 2 is a schematic microscopic view of a portion of a nanodiamond showing the structure of chemical entities attached to the surface of the nanodiamond.

FIG. 3 is an illustration of the entities of first part of chemical reactions for creating coated nanodiamonds according to the present invention.

FIG. 4 is an illustration of the entities of the second part of chemical reactions for creating coated nanodiamonds according to the present invention.

FIG. 5 is an illustration of test plates used for testing the effectiveness of the present invention.

### DETAILED DESCRIPTION OF THE INVENTION

### Theory

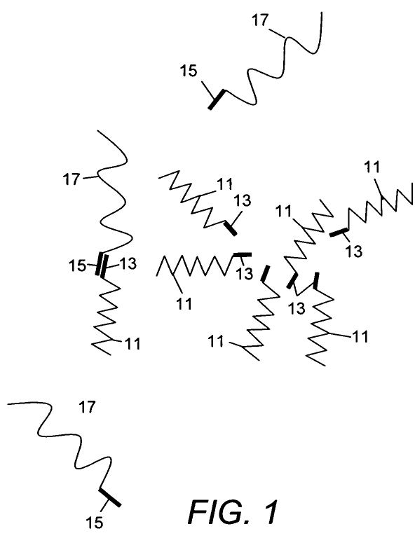

FIG. 1 is a schematic illustration of how molecules react under normal prior art conditions.

As stated in the “Background of the Invention”, chemical functional groups, antimicrobials AM 11 here for this description, typically in solution, randomly orient themselves and by random chance align in the proper orientation to have an active chemical site 13 of the AMs make contact with the proper active chemical site 15 of a molecule in a microbe 17.

If these active sites 13 are hidden inside a clump of molecules 11 (shown in the center of the figure) or otherwise inaccessible, the chances that the active sites 13 make contact another active site 15 of the microbe is reduced. It is better if the active sites are exposed.

When enough of these reactions occur, the reaction may cause the microbe to cease functioning. In one case it may cause them to stop their pathogenic function. In another case, the AMs may kill the microbe. This is the basis for fighting pathogens with antimicrobial chemicals.

These may be by numerous different mechanisms. One such mechanism is to impede a chain reaction which creates cell walls. This causes offspring to have weak cell walls making them more vulnerable to attack by the body's natural defenses such as macrophages.

Other mechanisms attack the microbe's ability to reproduce, or attack the energy producing mitochondria. Some AMs may use a combination of these mechanisms.

Since each of these are based upon the random motion of molecules in solution, the chances that an active site of a molecule having the proper orientation makes contact with an active site of the proper molecule of the microbe is a matter of chance. The greater the number of molecules and active sites in solution, the greater the chances of the desired chemical bindings between active sites. Therefore, by exposing and holding the active sites of the AMs outward in an exposed, fixed orientation and gradually varying the orientations across a surface, there will be an orderly array of exposed active sites.

This may be due to the fact that enough active sites on antimicrobial molecules must make contact with active sites on critical molecules inside of a microbe to cause the microbe to become deactivated.

Molecules flowing in a solution are randomly dispersed and oriented. Also, since AMs flow in solution to attach to active sites on the microbe, it is important to have a large amount of the AMs in solution, increasing the local concentration and the potential of attaching to an active site on a microbe molecule.

Also, the orderly arrangement of active sites must be able to move to meet up with the molecules of the microbe to interact with the active sites of these molecules. Therefore, this orderly arrangement must be mobile.

Foreign objects in the body are identified by the body's immune system and either destroyed or ejected from the body. The immune cells of the body may seek out and kill, or engulf and carry foreign objects out of the body. This would greatly reduce the efficacy of any drug introduced into the body which is recognized as a foreign substance.

The body ignores particles which are 10 Nanometers (nm.) or smaller. This may be due to the fact that there are many naturally occurring objects in the body fluids which are 10 nm. or smaller.

Nanodiamonds (“ND”) are diamonds which are 6 nm or smaller. These are typically produced according to the process explained in U.S. Pat. Nos. 5,916,955 and 5,861,349 assigned to NanoBlox, Inc. issued June and January 1999 respectively. In this process, carbon is converted in an explosive process to create NDs in which the vast majority of the NDs produced are approximately 6 nm.

These can be cleaned to take any graphite off of the surface to result in pure NDs of about 5 nm in diameter.

NDs have been shown to stabilize suspensions and solutions and greatly increase solubility of substances in solutions.

NDs have also been known to be functionalized to attach fluorine groups to its surface. This was intended to alter the surface composition of the NDs, but not for the purposes similar to that of the present invention.

Since particles of 10 nm or less are allowed to freely pass in and out of microbes, it is believed that these may be perfect transport vehicles for many different AMs. Therefore AMs would be attached to the NDs to create an AM-ND complex.

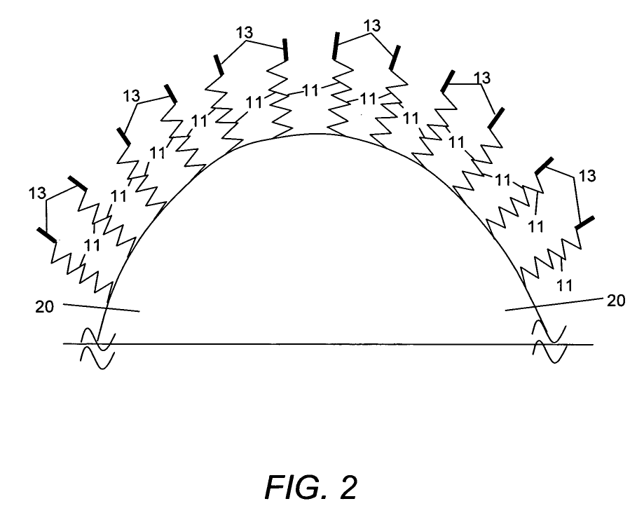

FIG. 2 is a schematic microscopic view of a portion of a nanodiamond showing the structure of chemical entities attached to the surface of the nanodiamond.

The ND 20 exhibits a spherical shape. Here one is covered with a plurality of chemical entities, which are antimicrobial chemical entities, the AMs 11. The AMs 11 are fixed in an orientation which extends them outwardly.

This causes the active sites 13 of each of the AMs 11 to be exposed and point outwardly. Since the surface of ND 20 is curved, as one moves along the surface in any direction, the orientation of the AMs 11 and their active sites 13 changes slightly, allowing a continuum of orientations for the active sites 13. Therefore, there is a greater chance of randomly oriented molecules to come in contact with an active site 13 of Am 11 having the proper orientation for reaction.

Therefore, if one were to supply an orderly arrangement of such AM molecules covering the surface of the NDs with the active sites facing outwardly, it is believed that the efficacy of the AMs would be greatly increased.

It was found, by extensive trial and error, that the efficacy of substances can be amplified by attachment to functionalized NDs. Modifying NDs has two major components. The first component is to cover the surface of the NDs with an intermediate compound. It was found that by replacing covalently attaching amine radicals to the exposed carbon chains of the NDs creates a platform which may then be used to attach other functional groups.

The second step would be to attach functional molecules and/or groups to the exposed amine groups.

Covalent Functionalization of Nanodiamond with Ethylenediamine.

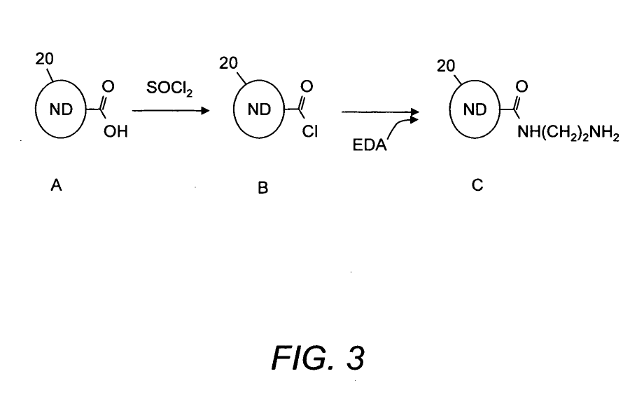

FIG. 3 is an illustration of the entities of first part of chemical reactions for creating coated nanodiamonds according to the present invention.

This wet chemistry procedure has been developed to selectively create amino functionalities at the surface of nanodiamond (ND) 20. Amino-terminated ND 20 has potential applications in medicine and as a component of novel versatile Nan composites. High temperature annealing in NH₃ atmosphere is non-selective resulting in a mixture of various surface functional groups including —NH₂, C═O—O—H, C═N etc. Wet chemistry rooted in mild conditions allows selectively create only —NH₂ surface functionalities-a strict requirement for the sophisticated applications mentioned above.

The sequence of reactions yielding the NH₂-terminated product attached to ND and Ethylenediamine (EDA) is presented below:

### Materials and Reagents.

Nanodiamond powder UD90 produced by detonation synthesis (FRPC “Altai”, Russia) was supplied by NanoBlox, Inc., USA. The powder was purified by oxidation in air [1] and then boiled with 35% wt. aqueous HCl under reflux for 24 hours to remove traces of metals and metal oxides. After removing the excess of HCl, ND powder was rinsed with distilled water and adjusted to a neutral pH and dried in the oven at 110° C. overnight.

In FIG. 3, this purified material is shown as entity A which is ND 20. This is used in subsequent functionalization. Here is can be seen that each ND entity A has a plurality of outwardly pointing COOH carboxyl groups covering the entire surface of the ND. (Only one is shown here for clarity.)

The reagents used were thionyl chloride purum ˜99.0% (Fluka), methanol anhydrous 99.8% (Sigma-Aldrich), tetrahydrofuran 99.85% ExtraDry (Acros Organics), ethylenediamine SigmaUltra (Sigma-Aldrich), N,N-dimethylformamide anhydrous, 99.8%. All reagents were used without additional purification.

### Synthesis of ND Acylchloride Derivative B of FIG. 3:

50 ml of SOCl₂ and 0.5 ml of anhydrous dimethylfomamide (DMF) (catalyst) were added to ˜1.5 g of carboxyl terminated ND particles (A) in round-bottom 100 ml flask. This was mixed with a Teflon-coated magnetic stirrer bar.

The flask was closed with a stopper and sonicated in an ultrasound bath until there were no visible chunks of nanodiamond.

The flask was then connected to a reflux condenser closed with a desiccating tube (Drierit) and heated under refluxing at 70° C. for 24 hours.

After cooling down to room temperature, excess of SOCl₂ was removed by vacuum distillation at a temperature of 50° C. to prevent its decomposition.

The content of the flask was then rinsed with anhydrous tetrahydrofuran (THF) (2 rinses 50 ml THF each); ND powder was allowed to precipitate. Excess THF was removed by decantation.

The flask was transferred into a desiccator with Drierit and left under vacuum at room temperature overnight to dry the ND acylchloride powder (B) of FIG. 3.

### Synthesis of ND Amino Derivative C of FIG. 3:

˜1.5 g of B was mixed with 50 ml of anhydrous EDA in round-bottom 100 ml flask. This was stirred with a Teflon-coated magnetic stirrer bar

The NDs now have EDA molecules covalently attached to them. The NDs have a spherical geometry. The EDAs are attached to the outermost surface making them the most exposed portion of the complex.

The functional groups and/or specific molecules may now be attached to each EDA of each ND-EDA complex.

### Step 2—Functionalization

The second step would be to attach functional molecules and/or groups to the exposed amine groups.

Covalent Functionalization of Aminated Nanodiamond with Antimicrobials.

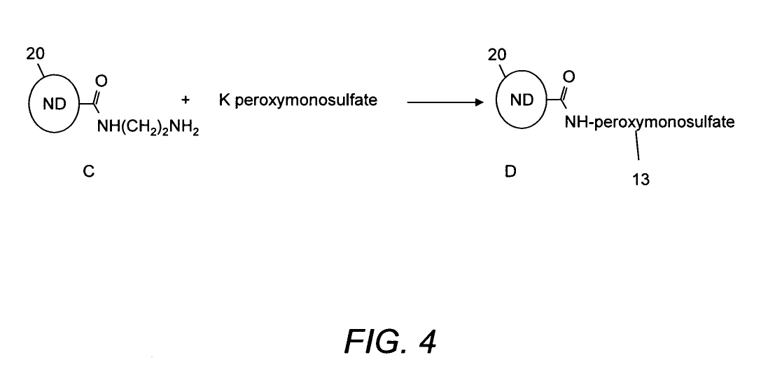

FIG. 4 is an illustration of the entities of the second part of chemical reactions for creating coated nanodiamonds according to the present invention.

This wet chemistry procedure has been developed to selectively create antimicrobial functionalities at the surface of nanodiamond (ND). Amino-terminated ND has potential applications in medicine and as a component of novel versatile nanocomposites. High temperature annealing in NH₃ atmosphere is non-selective resulting in a mixture of various surface functional groups including —NH₂, C═O—O—H, C═N etc. Wet chemistry rooted in mild conditions allows selectively create only —NH₂ surface functionalities-a strict requirement for the sophisticated applications mentioned above.

The sequence of reactions yielding the AM-terminated product attached to the aminated NDs.

### Materials and Reagents.

Aminated nanodiamond powder was reacted with K peroxymono sulfate (Fisher) and traditional antimicrobial moieties by salt synthesis.

The reagents used were thionyl chloride purum ˜99.0% (Fluka), methanol anhydrous 99.8% (Sigma-Aldrich), tetrahydrofuran 99.85% ExtraDry (Acros Organics), ethylenediamine SigmaUltra (Sigma-Aldrich), N,N-dimethylformamide anhydrous, 99.8%. All reagents were used without additional purification.

### Synthesis of ND Biofunctional Derivative D of FIG. 4:

In this part of the reaction, 5 grams of antimicrobial moieties and 0.5 ml of anhydrous dimethylfomamide (DMF) (catalyst) were added to ˜25 g of aminated ND particles (C) of FIG. 3 in round-bottom 200 ml flask. These are the NDs 20 having attached amine groups. This was mixed with a Teflon-coated magnetic stirrer bar.

The flask was closed with a stopper and sonicated in an ultrasound bath until there were no visible chunks of nanodiamond.

The flask was then connected to a reflux condenser closed with a desiccating tube (Drierit) and heated under refluxing at 70° C. for 24 hours.

After cooling down to room temperature, excess of liquid was removed by vacuum distillation at a temperature of 50° C. to prevent its decomposition.

The content of the flask was then rinsed with anhydrous tetrahydrofuran (THF) (2 rinses 50 ml THF each); ND powder was allowed to precipitate. Excess THF was removed by decantation.

The flask was transferred into a desiccator with Drierit and left under vacuum at room temperature overnight.

Freeze drying overnight produced the final bio-functional powder entity D of FIG. 4. Here it can be seen that NH-peroxymonosulfate is the antimicrobial attached to the ND 20. It has a reactive sites not shown here which are each numbered 13 in FIG. 2.

This has been described using terminal amine groups as the preferred embodiment. However, or chemical entities may be used as well. For example, the surface molecules on the nanodiamonds could be terminated with COOH groups. These will be replaced with the drug which is to be enhanced.

### Antimicrobials

A number of different antimicrobial functional groups may be attached to the EDA groups. In this example, potassium monopersulfate was used with the antimicrobial. Formulation of potassium monopersulfate is described in U.S. Pat. No. 7,090,820 B2 Martin, issued Aug. 15, 2006. It is understood that the present invention includes numerous different functional groups to enhance reactivity and efficacy.

Another preferred antimicrobial intended to be used with the present invention is: peroxymonosulfate triple salt and antimicrobial/antifungal chemistries as described in U.S. Pat. Nos. 7,090,820 and 4,131,672 issued Aug. 15, 2006 and Jan. 26, 1978 respectively.

Some other anti-microbial agents that can be used with the present invention include Cipro (Phizer), fluoroquinilone, Amoxycillin, 2-bromo-2-nitropropane-1,3-diol (for example, Canguard® 409 made by Angus Chemical Co., Buffalo Grove, Ill. 60089) and 3,5-dimethyltetrahydro-1,3,5-2H-thiazine-2-thione (for example, Nuosept® S made by Creanova, Inc., Piscataway, N.J. 08855 or Troysan®. 142 made by Troy Chemical Corp., West Hanover, N.J. 07936).

Other solid anti-microbial agents include N-(trichloromethyl)-thiop-hthalimide (for example, Fungitrol® 11 distributed by Creanova, Inc.), butyl-p-hydroxy-benzoate (for example, Butyl Parabens®. made by International Sourcing Inc., Upper Saddle River, N.J. 07458), diiodomethyl-p-tolysulfone (for example, Amical®. WP made by Angus Chemical Co.), and tetrachloroisophthalonitrile (for example, Nuocide® 960 made by Creanova, Inc.).

Drugs such as azithromycin, penicillin, clarithromycin, etc can be bound to the aminated and/or functionalized nanodiamond to increase efficacy as well.

Metals such as silver, copper and zinc and their metal ions also have anti-microbial properties. Silver ions have widespread effect as an anti-microbial agent. For example, silver ions may be effective against bacteria such as Escherichia coli and Salmonella typhimurium, and mold such as Aspergillus.

Sources of silver for functional groups for anti-microbial use include metallic silver, silver salts and organic compounds that contain ionic silver. Silver salts may include for example, silver carbonate, silver sulfate, silver nitrate, silver acetate, silver benzoate, silver chloride, silver fluoride, silver iodate, silver iodide, silver lactate, silver nitrate, silver oxide and silver phosphates. Organic compounds containing silver may include for example, silver acetylacetonate, silver neodecanoate and silver ethylenediaminetetraacetate in all its various salts.

Silver containing zeolites (for example, AJ10D containing 2.5% silver as Ag(I), and AK10D containing 5.0% silver as Ag(I), both distributed by AgION™ Tech L.L.C., Wakefield, Mass. 01880) are of particular use. Zeolites are useful for functional groups because when carried in a polymer matrix they may provide silver ions at a rate and concentration that is effective at killing and inhibiting microorganisms without harming higher organisms.

Silver containing zirconium phosphate (for example, AlphaSan® C 5000 containing 3.8% silver provided by Milliken Chemical, Spartanburg, S.C. 29304) is also particularly useful. In general zirconium phosphates act as ion exchangers. However, AlphaSan® C 5000 is a synthetic inorganic polymer that has equally spaced cavities containing silver, wherein the silver provides the anti-microbial effects. Silver zirconium phosphates are typically incorporated into powder coatings between 0.1 and 10 percent by weight and particularly 0.5 to 5 percent by weight of the total powder coating formulation.

Using AMs listed above in combination with NDs, the present invention greatly increases efficacy in fighting microbes. Below are microbes which may be reduced or stopped with greater efficacy.

Bacteria

The present invention, using nanodiamonds to enhance the effectiveness of the above-referenced AMs against bacteria such as: Bacillus cereus, Campylobacter jejuni, Chlamydia psittaci, Clostridium perfringens, Listeria monocytogenes, Shigella sonnei, Streptococcus pyogenes, Helicobacter pylori, Campylobacter pyloridia, Klebsiella pneumoniae, Escherichia coli, Salmonella typhimurium, Salmonella choleraesuis, Pseudomonas aeruginosa, Staphylococcus aureus, MRSA (Methicillin-Resistant Staphlococcus aureus), Staphylococcus epidermis and VRE (Vancomycin-Resistant Enterococci faecalis).

Viruses

It is also effective at increasing efficacy against viruses such as:

Hepatitis A (HAV), Hepatitis B (HBV), Hepatitis C(HCV), HIV-1 (AIDS Virus), Influenza A, Norovirus (Norwalk-like viruses) and RSV (Respiratory syncytial virus).

Fungi

It is also effective at increasing efficacy against fungi such as:

Candida albicans and Trichophytou mentagrophytes.

### Method of Delivery

There are various known methods of introducing the ND-AM complexes into the body of the patient. For example, the most obvious would be in a pill or liquid form which the subject ingests. This is only allowable for drugs which are not effected by the acids of the digestive tract.

The ND-AM complexes may injected, administered by air gun, nose spray, be inhaled, or used as a suppository.

The ND-AM complexes may be used as a disinfectant as an air spray, applied to the hands, or incorporated into materials around the patient, such as sheets and bedding.

They may also be incorporated into medical disposables, such as surgical drapes, bandages and disposable coverings.

The ND-AM complex may be used to coat the woven or non-woven fabrics. These may be for disposable or non-disposable fabrics. The ND-AM complexes may also be incorporated into the actual fibers used to create the woven or non-woven fabrics; and, includes but is not limited to applications in producing sutures, wound dressings, and medical cable coatings.

### Test Results

NDs coated with AMs showed exceptional results in fighting microbes. The coated NDs showed significantly enhanced efficacy when compared to the AMs used alone.

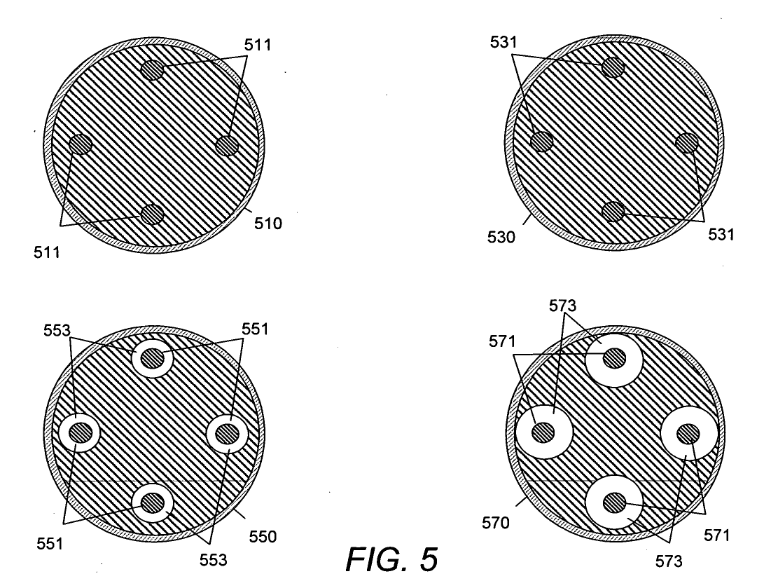

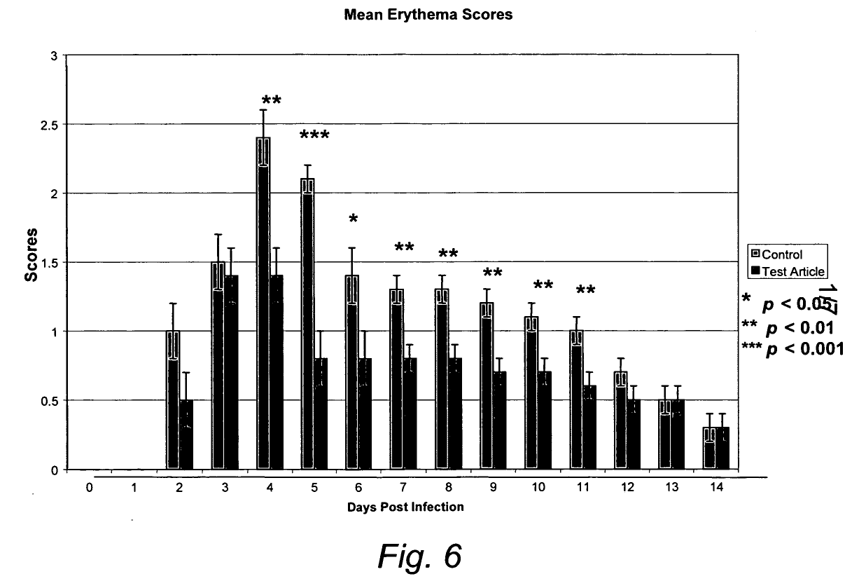

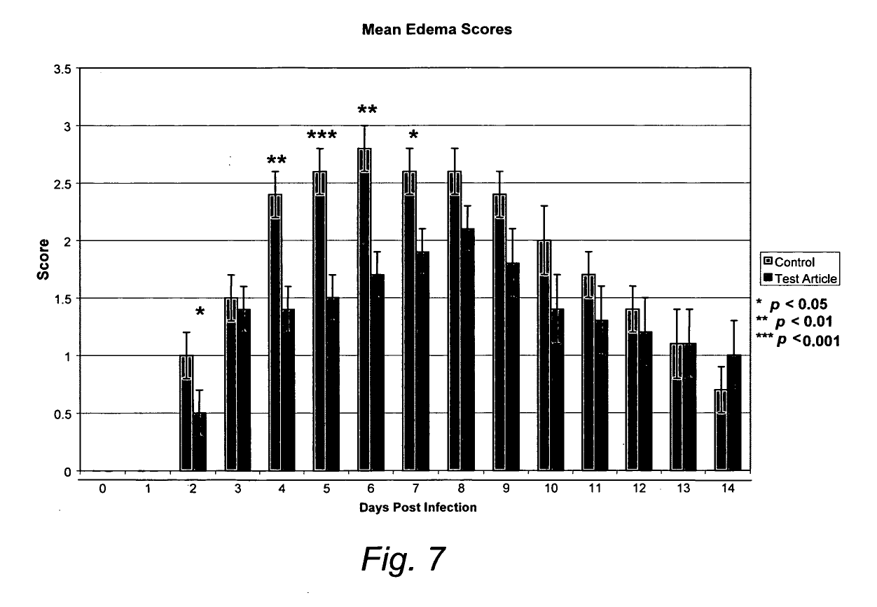

In FIG. 5 an illustration of the test results are shown. Three petri disks are shown filled with agar gel. Staphlococcus Aureus was previously grown evenly across the surface of the gels.

Sixteen approximately ¼″ diameter discs were fabricated from of DuPont Sontara 8801 non-woven fabric.

Four disks 511 were placed on the surface of the agar in dish 510.

Four disks 531 were coated with nanodiamond powder (A of FIG. 3) and were placed on the surface of the agar in dish 530.

Four disks 551 were coated with potassium monopersulfate, the antimicrobial described above and were placed on the surface of the agar in dish 550.

Four disks 571 were coated with potassium monopersulfate used as the antimicrobial attached to, and covering the entire surface of the NDs being entity D of FIG. 5 described above and were placed on the surface of the agar in dish 570.

The plates were all please in the same environment overnight, then examined.

Plate 510, 530 had continuous growth of the bacteria and were not changed.

Plate 550 developed a clear area 553 around each disk 551 which was due to the destruction of the bacteria previous growing in this region. This area extended approximately 1/16″ away from each of the disks 551.

Plate 570 developed a clear area 573 around each disk 571 which was due to the destruction of the bacteria previous growing in this region. This area extended approximately ⅛″ away from each of the disks 551.

This indicates that the disks 511 and the disks with ND 531 did not have any effect on the microbes.

The antimicrobial on disks 553 killed bacteria a small distance away and the antimicrobial attached to ND on disks 575 killed bacteria a significantly larger distance away.

The surface are of a spherical surface increases surface area based upon the square of the radius. Therefore if a given number of molecules has to cover a larger surface area, the concentration drops proportionally. Therefore, the ND-AM complex kills at a significantly lower concentration. This shows increased antimicrobial efficacy.

Medical Foam

The nanodiamond enhanced particles shown as ND-AM entity D on FIG. 4, may be mixed with a thermoplastic, then pumped with air to create either closed cell, or open cell medical foam. As a liquid, the foam resembles suds. Many different known plastics may be used. For our example below, the foam was created from polyurethane. It was then hardened into a spongy-like foam material with antimicrobial properties. This foam will be useful as a medical wound dressing.

Animal Testing

Medical foam was created as indicated above then sent to a medical laboratory for testing. An animal study was performed on 20 mice by an independent lab testing authority.

This activated polyurethane foam was created with approximately almost 4% active material (ND-AM entity D on FIG. 4). The thickness of the used foam is 1.5 mm. This is referred to in the figures as the “Test Article”.

Wound areas having a diameter of about 4 mm. on each of the mice were infected by cultured Methycillin Resistant Staphlococcus Aureus (MRSA) infected with a 0.1 ml volume dose of inoculum which occurred only on Day 0.

There was also polyurethane foam without the ND-AM of the same size and thickness placed on the mice as a control for comparisons. This is referred to in the figures as the “Control”.

The foam was changed daily on each of the wounds, and the results tabulated.

The data is listed below. Table A shows Mean Erythema Scores of both the tested group and the control group for consecutive 14 days. This is shown graphically in FIG. 6.

Table B shows the Mean Edema Scores for the same groups for 14 consecutive days. This is shown graphically in FIG. 7.

Table C shows the Mean Wound Diameters for the same groups for 14 consecutive days. This is shown graphically in FIG. 8.

Table D shows the Average Time to Exfoliation for both groups.

Table E shows the Body Weights of the test groups over the test period.

### Other Test Results

TABLE A
Mean Erythema Scores

| | | Erythema Scores | | | | | | | | | | | | | | |
|---|---|---|---|---|---|---|---|---|---|---|---|---|---|---|---|---|
| | | (mean ± sem) | | | | | | | | | | | | | | |
| | | Day | Day | Day | Day | Day | Day | Day | Day | Day | Day | Day | Day | Day | Day | Day |
| Group | Treatment | 0 | 1 | 2 | 3 | 4 | 5 | 6 | 7 | 8 | 9 | 10 | 11 | 12 | 13 | 14 |
| 1 | Vehicle | 0 | 0 | 1.0 ± | 1.5 ± | 2.4 ± | 2.1 ± | 1.4 ± | 1.3 ± | 1.3 ± | 1.2 ± | 1.1 ± | 1.0 ± | 0.7 ± | 0.5 ± | 0.3 ± |
| | | | | 0.2 | 0.2 | 0.2 | 0.1 | 0.2 | 0.1 | 0.1 | 0.1 | 0.1 | 0.1 | 0.1 | 0.1 | 0.1 |
| 2 | Activated | 0 | 0 | 0.5 ± | 1.4 ± | 1.4 ± | 0.8 ± | 0.8 ± | 0.8 ± | 0.8 ± | 0.7 ± | 0.7 ± | 0.6 ± | 0.5 ± | 0.5 ± | 0.3 ± |
| | PU foam | | | 0.2 | 0.2 | 0.2** | 0.2*** | 0.2* | 0.2** | 0.1** | 0.1** | 0.1** | 0.1** | 0.1 | 0.1 | 0.1* |

TABLE B
Mean Edema Scores

| | | Edema Scores | | | | | | | | | | | | | | |
|---|---|---|---|---|---|---|---|---|---|---|---|---|---|---|---|---|
| | | (mean ± sem) | | | | | | | | | | | | | | |
| | | Day | Day | Day | Day | Day | Day | Day | Day | Day | Day | Day | Day | Day | Day | Day |
| Group | Treatment | 0 | 1 | 2 | 3 | 4 | 5 | 6 | 7 | 8 | 9 | 10 | 11 | 12 | 13 | 14 |
| 1 | Vehicle | 0 | 0 | 1.0 ± | 1.5 ± | 2.4 ± | 2.6 ± | 2.8 ± | 2.6 ± | 2.6 ± | 2.4 ± | 2.0 ± | 1.7 ± | 1.4 ± | 1.1 ± | 0.7 ± |
| | | | | 0.2 | 0.2 | 0.2 | 0.2 | 0.2 | 0.1 | 0.2 | 0.2 | 0.3 | 0.2 | 0.2 | 0.3 | 0.1 |
| 2 | Activated | 0 | 0 | 0.5 ± | 1.4 ± | 1.4 ± | 1.5 ± | 1.7 ± | 1.9 ± | 2.1 ± | 1.8 ± | 1.4 ± | 1.3 ± | 1.2 ± | 1.1 ± | 1.0 ± |
| | PU foam | | | 0.2* | 0.2 | 0.2** | 0.2*** | 0.2** | 0.2* | 0.2 | 0.3 | 0.3 | 0.3 | 0.3 | 0.3 | 0.3 |

TABLE C
Mean Wound Diameters

| | | Wound Diameters | | | | | | | | | | | | | | |
|---|---|---|---|---|---|---|---|---|---|---|---|---|---|---|---|---|
| | | (mean ± sem) | | | | | | | | | | | | | | |
| | | Day | Day | Day | Day | Day | Day | Day | Day | Day | Day | Day | Day | Day | Day | Day |
| Group | Treatment | 0 | 1 | 2 | 3 | 4 | 5 | 6 | 7 | 8 | 9 | 10 | 11 | 12 | 13 | 14 |
| 1 | Vehicle | 5.7 ± | 5.6 ± | 5.3 ± | 5.1 ± | 4.8 ± | 4.6 ± | 4.3 ± | 4.3 ± | 3.9 ± | 3.2 ± | 2.9 ± | 2.7 ± | 2.3 ± | 2.2 ± | 1.8 ± |
| | | 0.3 | 0.3 | 0.3 | 0.3 | 0.3 | 0.3 | 0.3 | 0.3 | 0.3 | 0.2 | 0.2 | 0.2 | 0.2 | 0.1 | 0.1 |
| 2 | Activated | 5.9 ± | 5.9 ± | 5.0 ± | 4.9 ± | 4.7 ± | 4.6 ± | 4.1 ± | 3.8 ± | 3.5 ± | 3.0 ± | 2.6 ± | 2.3 ± | 2.0 ± | 1.8 ± | 1.5 ± |
| | PU foam | 0.4 | 0.2 | 0.2 | 0.2 | 0.2 | 0.2 | 0.2 | 0.2 | 0.1 | 0.1 | 0.1 | 0.1* | 0.1 | 0.1* | 0.1* |

TABLE D
Average Time to Exfoliation

| | | Exfoliation |
|---|---|---|
| | | Time |
| | | (days) |
| | | Mean ± |
| Group | Treatment | SEM |
| 1 | Vehicle | 9.9 ± 0.8 |
| 2 | Activated PU Foam | 10.8 ± 0.6 |

*Statistically significant difference compared to the Group 1 vehicle control (p < 0.05)
**Statistically significant difference compared to the Group 1 vehicle control (p < 0.05)
***Statistically significant difference compared to the Group 1 vehicle control (p < 0.05)

TABLE E
Body Weights

| | | Body Weight (g) | | |
|---|---|---|---|---|
| | | (mean ± sem) | | |
| Group | Treatment | Day 0 | Day 7 | Day 14 |
| 1 | Vehicle | 24.6 ± 0.4 | 25.2 ± 0.6 | 25.8 ± 0.6 |
| 2 | Activated PU Foam | 23.2 ± 0.4 | 23.9 ± 0.6 | 24.4 ± 0.7 |

Wound infection is a major complication in diabetic patients. According to the American Diabetes Association, 25% of people with diabetes will suffer from a wound problem during their lifetime, and it has been estimated that lower limb amputations in diabetic patients account for >60% of all amputations performed.

Staphylococcus aureus (S. aureus) is the most common single isolate (76%) in diabetic wounds and foot ulcers and leads to alterations in wound healing. Wound infection can also result in bacteremia or sepsis and is associated with high morbidity and mortality. In the United States S. aureus is the most common cause of skin and soft-tissue infections, as well as of invasive infections acquired within hospitals. Treatment of severe S. aureus infections is challenging, and the associated mortality rate remains high. S. aureus is a gram-positive bacterium that colonizes the skin and is present in the anterior nares in about 25-30% of healthy people. Over the last 40 years Methicillin-resistant S. aureus (MRSA) infections have become endemic in hospitals in the U.S. and worldwide. In 2002, the first clinical isolate of Vancomycin-resistant S. aureus (VRSA) was identified in a patient with diabetic foot ulcer. The progressive reduction of therapeutic efficacies of the available antibiotics underlines the need for the development of new therapeutic strategies for the treatment of infected wounds.

Therefore, the medical foam incorporating the enhanced antimicrobial would be very useful in countering the problems which diabetics encounter, especially since it was tested as effective against MRSA.

Even though this description was performed for a specific antimicrobial, it is believed that this applies to increasing the efficacy of other drugs and preparations. If these entities are used instead of the AM entities and attached to the surface of NDs, their efficacy will also increase.

Since other modifications and changes varied to fit particular operating requirements and environments will be apparent to those skilled in the art, the invention is not considered limited to the example chosen for the purposes of disclosure, and covers all changes and modifications which do not constitute departures from the true spirit and scope of this invention.

What is claimed is:

1. A method of enhancing efficacy of a drug having an active site, comprising the steps of:
a) acquiring a plurality of nanodiamond (ND) particles having a plurality of carbon chain surface molecules on its surface, the ND particles having a diameter of less than 10 nanometers;
b) covalently attaching a plurality of intermediate amine entities to the surface molecules of the ND particles, and
c) replacing at least a portion of the attached intermediate amine entities attached to the surface molecules of the ND particles with said drug molecules such that the active sites of said drug molecules point away from the ND particle exposing them for enhanced activity and enhanced drug efficacy.

2. The method of claim 1, wherein the drug molecules comprise:
antimicrobial agents.

3. The method of claim 1, wherein the drug molecules comprise:
peroxymonosulfate salts.

4. The method of a claim 1 wherein the drug molecules comprise molecules of one of the group consisting of:
fluoroquinilone, Amoxycillin, 2-bromo-2-nitropropane-1,3-diol, 3,5-dimethyltetrahydro-1,3,5-2H-thiazine-2-thione, N-(trichloromethyl)-thiop-hthalimide, butyl-p-hydroxy-benzoate, diiodomethyl-p-tolysulfone, and tetrachloroisophthalonitrile, azithromycin, penicillin and clarithromycin.

5. A method of enhancing the efficacy of drug molecules comprising the steps of:
a) acquiring nanodiamond (ND) particles having carbon chain surface molecules created by a detonation process with the majority of the particles having a diameter of less than 10 nm;
b) exposing ND particles to air for a period of time to oxidize the surface molecules;
c) boiling the oxidized ND particles in aqueous hydrochloric acid to remove metal and metal oxides from the surface of the ND particles to result in surface molecules of the ND particles to be terminated with carboxyl groups (A);
d) synthesizing an acylchloride derivative (B) from the ND particles having surface molecules terminated with carboxyl groups;
e) synthesizing an ND amino derivative from the surface molecules of the ND particles terminated with carboxyl groups;
f) replacing the terminal EDA entities with said drug molecules to result in functionalized ND particles (D) exhibiting enhanced efficacy of said drug molecules.

6. The method of claim 5 wherein step of synthesizing an acylchloride derivative, comprises the steps of:
mixing SOCl₂ with anhydrous dimethylformamide (DMF) and the carboxyl terminated surface molecules of the ND particles (A);
heating the mixture from the previous step;
rinsing the mixture with anhydrous tetrahyrofuran to result in a rinsed mixture;
drying the rinsed mixture to recover a powder which is the ND acylchloride derivative (B).

7. The method of claim 5, wherein step of synthesizing an ND amino derivative, comprises the steps of:
adding anhydrous ethylenediamine, NHCH₂CH₂NH₂, (EDA) to the acylchloride derivative of the ND particles (B); and
mixing the EDA and the acylchloride derivative to result in ND particles having surface molecules covalently attached to EDA (C) over the surface of the ND particles.

8. The method of claim 5, further comprising the step of:
administering the functionalized ND particles (D) to a patient by injection.

9. The method of claim 5, further comprising the step of:
administering the functionalized ND particles (D) to a patient by compressed air gun.

10. The method of claim 5, further comprising the step of:
administering the functionalized ND particles (D) to a patient as a nose spray.

11. The method of claim 5, further comprising the step of:
administering the functionalized ND particles (D) to a patient as a suppository.

12. The method of claim 5, further comprising the step of:
incorporating the functionalized ND particles (D) into fibers of nonwoven fabrics used in the medical industry.

13. The method of claim 5, further comprising the step of:
incorporating the functionalized ND particles (D) into threads of woven fabrics used in the medical industry.

14. The method of claim 5, further comprising the step of:
incorporating the functionalized ND particles (D) into fibers of nonwoven fabrics used in surgical drapes.

15. The method of claim 5, further comprising the step of:
incorporating the functionalized ND particles (D) into fibers of nonwoven fabrics used in disposable surgical garments.

16. The method of claim 5, further comprising the step of:
incorporating the functionalized ND particles (D) into fibers of nonwoven fabrics used in disposable wound care dressings.

17. The method of claim 5, further comprising the step of:
incorporating the functionalized ND particles (D) into the threads of woven fabrics used for clothing exhibiting antimicrobial properties.

18. The method of claim 5, further comprising the step of:
incorporating the functionalized ND particles (D) into the threads of woven fabrics used to make clothing resistant to microbial growth and unpleasant odors.

19. An enhanced efficacy drug complex created by performing the process comprising the steps of:
a) acquiring nanodiamond (ND) particles having carbon chain surface molecules created by a detonation process with the majority of the particles having a diameter of less than 10 nm;
b) exposing ND particles to air for a period of time to oxidize the surface molecules;
c) boiling the oxidized ND particles in aqueous hydrochloric acid to remove metal and metal oxides from the surface of the ND particles to result in surface molecules of the ND particles to be terminated with carboxyl groups (A);
d) synthesizing an acylchloride derivative (B) from the ND particles having surface molecules terminated with carboxyl groups;
e) synthesizing an ND amino derivative from the surface molecules of the ND particles terminated with carboxyl groups;
f) replacing the terminal EDA entities with said drug molecules to result in functionalized ND particles (D) exhibiting enhanced efficacy of said drug molecules.

20. The enhanced efficacy drug complex of claim 19 wherein step of synthesizing an acylchloride derivative, comprises the steps of:
mixing SOCl₂ with anhydrous dimethylformamide (DMF) and the carboxyl terminated surface molecules of the ND particles (A);
heating the mixture from the previous step;
rinsing the mixture with anhydrous tetrahyrofuran to result in a rinsed mixture;
drying the rinsed mixture to recover a powder which is the ND acylchloride derivative (B).

21. The enhanced efficacy drug complex of claim 19, wherein step of synthesizing an ND amino derivative, comprises the steps of:
adding anhydrous ethylenediamine, NHCH₂CH₂NH₂, (EDA) to the acylchloride derivative of the ND particles (B); and
mixing the EDA and the acylchloride derivative to result in ND particles having surface molecules covalently attached to EDA (C) over the surface of the ND particles.

22. The enhanced efficacy drug complex of claim 19 wherein the step of replacing the terminal EDA entities with said drug molecules comprises the step of:
replacing the terminal EDA entities with said drug molecules selected form the group consisting of:
fluoroquinilone, Amoxycillin, 2-bromo-2-nitropropane-1,3-diol, 3,5-dimethyltetrahydro-1,3,5-2H-thiazine-2-thione, N-(trichloromethyl)-thiop-hthalimide, butyl-p-hydroxy-benzoate, diiodomethyl-p-tolysulfone, and tetrachloroisophthalonitrile, azithromycin, penicillin and clarithromycin.

23. The method of claim 5, further comprising the step of:
incorporating the functionalized ND particles (D) into a liquid plastic;
foaming the liquid plastic and functionalized ND particles (D) allowing the liquid plastic and ND particles (D) to harden into a medical foam for use in wound care.
